(19) United States
(12) Patent Application Publication
(10) Pub. No.: US 2026/0083693 A1
(43) Pub. Date: Mar. 26, 2026

(54) PHARMACEUTICAL COMPOSITION FOR THE ACUTE TREATMENT OF MIGRAINE AND OTHER FORMS OF HEADACHE

(71) Applicant: CSS PHARMA L.L.C., Delray Beach, FL (US)

(72) Inventors: Guillermo Antonio GARCIA PITTALUGA, Ciudad San Nicolas (AR); Julio César VILLAMIL TORRES, Bogota (CO); Sankar SHANMUGAM, Delray Beach, FL (US); Camilo REY FERRO, Weston, FL (US)

(21) Appl. No.: 18/894,711

(22) Filed: Sep. 24, 2024

Related U.S. Application Data
(63) Continuation of application No. PCT/US2024/047972, filed on Sep. 23, 2024.

(51) Int. Cl. A61K 31/192; A61K 9/00; A61K 9/08; A61P 25/06

(52) U.S. Cl. CPC A61K 31/192; A61K 9/0043; A61K 9/08; A61P 25/06

(57) ABSTRACT

A pharmaceutical composition for intranasal administration and manufacturing method of preparations containing a combination of ibuprofen and ketoprofen at a fraction of the dose required when the same active ingredients are administered orally, useful in the treatment of migraine and other forms of headache.

### CROSS-REFERENCE TO RELATED APPLICATIONS

This application claims priority from U.S. provisional application No. 63/595,435 filed on Nov. 11, 2023, and is a continuation of international application number PCT/US2024/047972 filed on Sep. 23, 2024, which are both incorporated herein by reference in their entirety.

### FIELD OF THE INVENTION

The present invention relates to pharmaceutical compositions and manufacturing methods of preparations containing a combination of two nonsteroidal anti-inflammatory drugs (NSAIDs), useful in the acute treatment of migraine and other forms of headache. More particularly, the present invention is related to an isotonic solution containing a combination of ibuprofen and ketoprofen which, when administered through the intranasal route, is able to reach the desired pharmacological effect with only a fraction of the dose required when the same active ingredients are administered through the oral route. This dose reduction allows a minimization of the dose-dependent adverse effects associated to these active ingredients, enabling their use in a group of patients who do not tolerate NSAIDs at the currently approved therapeutic dosages.

### BACKGROUND OF THE INVENTION

Migraine is a common neurological condition characterized by recurrent attacks of moderate to severe throbbing and pulsating pain on one side of the head, in most of the cases accompanied by other symptoms such as nausea, vomiting, photophobia, phonophobia, osmophobia and allodynia. The pain is caused by the activation of nerve fibers within the wall of brain blood vessels traveling inside the meninges (three layers of membranes protecting the brain and spinal cord).

Migraine is a debilitating condition, representing the second leading cause of disability globally. In a recent epidemiological study in the United States (US), 19.2% of individuals have self-reported a migraine, with 15.8% reporting at least one monthly headache day over a 3-month period (Martin, V., Hoekman, J., Aurora, S., & Shrewsbury, S. (2021 June 2). Nasal delivery of acute medications for migraine: The upper versus lower nasal space. Journal of Clinical Medicine, 10(11), 2468. doi: 10.3390/jcm10112468).

Migraine treatment is aimed at 1) relieving symptoms and 2) preventing additional attacks. According to that, drug therapy for migraine is divided into 1) acute treatment and 2) preventive treatment. Acute medications are taken as soon as symptoms occur to relieve pain and restore function. Preventive treatment involves taking medicines daily to reduce the severity of future attacks or keep them from happening.

Acute treatment for migraine may include any of the following drugs:

Triptan drugs are the preferred choice to ease migraine from moderate to severe. Triptans can be administered whether orally or intranasally, and act by selectively binding to the serotonin receptors 5-HT1B and 5-HT1D. Binding to the vascular 5-HT1B receptors leads to vasoconstriction of the cranial arteries, which painfully dilate during a migraine attack.

Triptans have cardiovascular side effects and are contraindicated during pregnancy and when a selective serotonin reuptake inhibitor (SSRI) is being administered. Furthermore, only 30% to 40% of migraineurs are pain-free two hours after the administration of triptans. Of those who do respond, one in three will experience a migraine recurrence within 24 hours.

NSAIDs which reduce inflammation and alleviate pain.

Ergot derivative drugs. These are most effective during the early stages of migraine and its use is limited because of its side effects which include vomiting.

Non-prescription analgesics or over-the-counter drugs such as ibuprofen, aspirin, or acetaminophen can help to ease the pain. However, those are only useful for less severe migraine headache.

Narcotics are prescribed briefly to relieve pain. These drugs should not be used to treat chronic headaches because of issues related to narcotic abuse.

Combination analgesics involve a mix of drugs such as acetaminophen plus caffeine and/or a narcotic for migraine that may be resistant to simple analgesics.

Nausea relief drugs can ease queasiness brought on by various types of headaches. (National Institute of Neurological Disorders and Stroke. (2023 June 2). National Institute of Health. Retrieved from Migraine: https://www.ninds.nih.gov/health-information/disorders/migraine).

As mentioned before, NSAIDs are one of the preferred therapeutic alternatives for the acute treatment of migraine and its common symptoms.

NSAIDs act by inhibiting the enzyme cyclooxygenase (COX), Cyclooxygenase is required to convert arachidonic acid into thromboxanes, prostaglandins, and prostacyclins. The therapeutic effects of NSAIDS are attributed to the lack of these eicosanoids. Specifically, thromboxanes play a role in platelet adhesion, prostaglandins cause vasodilation, increase the temperature set-point in the hypothalamus, and play a role in anti-nociception.

There are two cyclooxygenase isoenzymes, COX-1 and COX-2. COX-1 gets constitutively expressed in the body, and it plays a role in maintaining gastrointestinal mucosa lining, kidney function, and platelet aggregation. Most of the NSAIDs are nonselective and inhibit both COX-1 and COX-2. However, COX-2 selective NSAIDs (ex. celecoxib) only target COX-2 and therefore have a different side effect profile.

NSAIDs are typically divided into groups based on their chemical structure and selectivity: salicylates (aspirin), non-acetylated salicylates (diflunisal, salsalate), propionic acid derivatives (naproxen, ibuprofen), acetic acid derivatives (diclofenac, indomethacin), enolic acid derivatives (meloxicam, piroxicam) anthranilic acid derivatives (meclofenamate, mefenamic acid), naphthylalanine (nabumetone), and selective COX-2 inhibitors (celecoxib, etoricoxib). (Ghlichloo, I., & Gerriets, V. (2019) Nonsteroidal anti-inflammatory drugs (NSAIDs). Treasure Island (FL): StatPearls Publishing).

Two of the most frequently utilized NSAIDs from the propionic acid derivative class are ibuprofen and ketoprofen. When administered orally at currently approved dosages, both NSAIDs achieve maximum plasma concentrations in the range of micrograms per milliliter (μg/mL). For instance, oral administration of ibuprofen at a dose of 100 mg results in a peak plasma concentration of 9.5 μg/mL at 116 minutes (Hattrem, Dille, Seternes, Ege, & Draget, 2018). Similarly, oral administration of 400 mg of ibuprofen yields a maximum plasma concentration of 32.8 μg/mL at 90 minutes (Dewland, Reader, & Berry, 2009). In contrast, oral administration of ketoprofen at a dose of 50 mg leads to a peak plasma concentration of 2.6 μg/mL at 134 minutes (Upton, Williams, Guentert, Buskin, & Riegelman, 1981).

Despite the proved efficacy of NSAIDs in the acute treatment of migraine, their use is often limited by their well-known adverse effects, which are related to the relatively high plasma concentrations achieved post-administration and can affect the gastric mucosa, renal system, cardiovascular system, hepatic system, and hematologic system.

Regarding the gastric adverse effects, due to the inhibition of COX-1, NSAIDs blocks the synthesis of prostaglandins that protect the gastric mucosa. The damage is more likely in a patient that has a prior history of peptic ulcers.

Regarding the renal adverse effects, COX-1 and COX-2 facilitate the production of prostaglandins that play an important role in renal hemodynamics. In a patient with renal dysfunction, the reduction of these prostaglandins due to the treatment with NSAIDs can lead to complications including acute renal dysfunction, fluid and electrolyte disorders, renal papillary necrosis, and nephrotic syndrome/interstitial nephritis.

Regarding the cardiovascular adverse effects, these include MI, thromboembolic events, and atrial fibrillation.

On the other hand, although hepatic adverse effects are less common, NSAIDs has been associated with risk of hepatotoxicity, (raised aminotransferase levels) and liver related hospitalization.

Hematologic adverse effects are possible, particularly with nonselective NSAIDs due to their antiplatelet activity. This antiplatelet effect typically only poses a problem if the patient has a history of GI ulcers, diseases that impair platelet activity (hemophilia, thrombocytopenia, von Willebrand, etc.), and in some perioperative cases.

Other minor adverse effects include anaphylactoid reactions that involve the skin and pulmonary systems, like urticaria and aspirin-exacerbated respiratory disease. (Ghlichloo & Gerriets, 2019).

Given the usefulness of NSAIDs in the chronic treatment of migraine, but also given their usage limitations due to their adverse effects, there is a need for a NSAIDs containing dosage form with and enhanced adverse effects profile, which enable their use in patients who usually do not tolerate them.

One of the strategies proposed to reduce the adverse effects of the acute treatment of migraine has been the use of alternative routes of administration requiring less dosage, Since NSAID-related adverse effects are dose-depending, a reduction of the dosage could lead to a reduction in the adverse effects and an enhanced safety profile. (Vonkeman, H., & van de Laar, M. (2010 February). Nonsteroidal anti-inflammatory drugs: adverse effects and their prevention. Seminars Arthritis and Rheumatism, 39 (4), 294-312. doi: 10.1016/j.semarthrit.2008.08.001).

The most explored alternative administration routes include injection (subcutaneous (SC), intramuscular (IM), or intravenous (IV)), topical (transdermal), and inhalation (nasal and pulmonary). Among these alternatives, one of the most interesting has been the nasal route of administration, because it offers several advantages including at-home administration, non-invasiveness, and easy self-administration as well as avoidance of drug degradation in the GI tract and first-pass metabolism (similar to injection), which allows for enhanced bioavailability, reduction of the required dosage and reduction of systemic side effects without the use of a needle.

Nasal route is especially striking in the acute treatment of migraine since it enables the possibility to reach directly the innervation of the trigeminal nerve, which has been considered a common denominator in headache pathophysiology. Thalamic trigeminovascular neurons contribute to migraine pain and mediate many migraine-associated symptoms, such as photophobia, phonophobia, osmophobia and allodynia. (Martin, Hoekman, Aurora, & Shrewsbury, 2021).

U.S. Pat. No. 6,090,368 discloses a pharmaceutical nasal spray containing an effective amount of ketorolac, in the form of tromethamine, as a NSAID for the treatment of pain such as postoperative pain, cancer pain and migraine headache. The concentration of ketorolac in the preparation ranges from 2% to 15% (w/v) and it is accompanied by a bio adhesive cellulosic polymer.

Commercially available ketorolac tromethamine nasal spray is presented as a solution containing 15.75% (w/v) of ketorolac tromethamine, equivalent to 10.68% (w/v) of ketorolac. The product is designed to be administered as a 100 μL spray into each nostril, for a total of 31.5 mg of ketorolac tromethamine per dose. U.S. Food and Drug Administration. (2023 May 6). Drug Databases. Retrieved May 6, 2023, from Drugs@FDA: FDA-Approved Drugs: https://www.accessdata.fda.gov/scripts/cder/daf/index.cfm. Compared to the approved oral dose of ketorolac tromethamine, 5 and 10 mg film-coated tablets and 30 mg sublingual tablets, in this case intranasal administration route failed in getting the objective of reducing the required dosage of Ketorolac tromethamine, since intranasal dose was found to be even higher than the corresponding oral dose.

U.S. Pat. No. 11,026,882 B2 discloses a method of treating pain associated with migraine and pain or inflammation associated with temporomandibular disorder (TMD) comprising the topical administration of a sustained release composition containing from about 0.5% (w/w) to about 5% (w/w) ketoprofen or from about 20 mg to about 200 mg of ketoprofen in a unit dosage form. The application of said composition results in a peak plasma concentration of ketoprofen, at three hours, that is at most about 450 ng/ml, maintained between about 50 ng/ml and 150 ng/ml for up to 24 hours.

In this case, even assuming the lower disclosed dose per unit dosage form of ketoprofen, which is 20 mg, it corresponds to 160% of the content of the minimum approved oral dose form of the same active ingredient which is 12.5 mg. (U.S. Food and Drug Administration, 2023).

Other documents referring the use of alternative routes of administration of NSAIDs for the treatment of migraine and other forms of pain are listed below, however, none of these documents suggest the reduction of the required dose of the NSAID compared to their corresponding oral approved dose.

United States Patent Applications US 2013/0109674 A1 and US 2019/0105290 A1 discloses a method of treating migraine headache in a human subject by means of the topical administration of a NSAID containing composition to the area of the sternocleidomastoideole at the temporomandibular joint of the subject. The preferred pharmaceutical dosage forms are creams and ointments and the concentration of the NSAIDs into the preparations varies depending on the selected NSAID. For ibuprofen, the concentration ranges from 10% to 30% (w/w), while for ketoprofen, the concentration ranges from 1% to 10% (w/w).

United States Patent Applications US 2003/0119892 A1 and US 2007/0112052 A9 discloses methods and compositions for treating a mammal suffering from headache pain comprising the topical application of a nonsalicylate NSAID to a keratinized skin surface. The NSAID is formulated in the form of a cream or a patch.

U.S. Pat. No. 8,822,537 B2 discloses a topical composition, specifically an oil-in-water emulsion, containing about 0.5% to about 15% (w/w) ketoprofen and about 2.5% to about 6% (w/w) oxybenzone with the objective of alleviating the pain, especially pain associated with migraine headaches.

United States Patent application US 2010/0099650 A1 discloses a composition for intranasal administration in the form of a spray, with the objective of treating mucosal inflammation associated with rhinitis, sinusitis, or both. The composition contains a decongestant and at least one additional therapeutic agent among which are anti-inflammatory agents such as the NSAIDs.

U.S. Pat. No. 7,473,432 B2 discloses a bilayer vesicular composition made of phosphatidylcholine and a surfactant. Said vesicles containing salts of an NSAID wherein said NSAID is diclofenac, ibuprofen or ketoprofen. The composition is designed to be administered topically, in the form of a non-occlusive patch, to treat peripheral pain or inflammation.

United States Patent application US 2008/0317684 A1 discloses a composition comprising and NSAID dissolved in a solvent system composed by at least two solvent alcohols in a one single phase system. The concentration of the NSAID in the preparation ranges from about 12% to about 30% (w/w).

United States patent application US 2004/0138239 A1 and its corresponding U.S. Pat. No. 7,981,901 B2 disclose a method to directly apply a pharmacological active substance to a cerebral circulation through the topical application of a transdermal composition to a tender spot associated with the headache. The transdermal composition includes an active substance selected from the group consisting of xanthine derivative, vitamin B6 and angiotensin-converting enzyme, combined with a skin penetration enhancer, in the form of a cream, lotion, ointment, salve or paste. In an especially preferred composition, ketoprofen is added to the preparation in a concentration ranging from about 0.5% to about 50% (w/w).

Other documents refer to liquid intermediate compositions containing solubilized ketoprofen to be used in the preparation of a wide variety of pharmaceutical dosage forms. However, these documents do not suggest any reduction in the required dosage of ketoprofen, as they are limited to the currently approved dosages for oral administration which begins in 12.5 mg.

The United States patent U.S. Pat. No. 7,090,859 B2 discloses a liquid composition in which ketoprofen is solubilized in an aqueous media by using potassium bicarbonate in a ratio of 2.5 grams of ketoprofen to 1 gram of potassium bicarbonate.

On the other hand, U.S. Pat. No. 8,178,112 B2 discloses a liquid composition in which ketoprofen is solubilized in an aqueous media by using an alkali metal bicarbonate, reaching a concentration ranging from about 12.5 mg of ketoprofen per 5 mL to about 150 mg of ketoprofen per 5 mL. The alkali metal bicarbonate can be sodium bicarbonate or potassium bicarbonate.

Based on this, it can be concluded that the minimum currently approved oral dosage of ketoprofen for the treatment of pain is 12.5 mg, therefore there is no product disclosed in the state of the art that contains less than 12.5 mg of ketoprofen per dosage unit.

Finally, other documents referring the use of the nasal route of administration to treat headache and migraine were found. In this case, the active substance is an anesthetic.

United States patent U.S. Pat. No. 6,432,986 B2 discloses methods, kits and compositions for inhibiting neurovascular disorders or a muscular headache, such as migraine, in a human patient, comprising dorsonasally administering of a long-lasting anesthetic.

### SUMMARY OF THE INVENTION

A pharmaceutical composition for intranasal administration and treatment of migraines and other headaches, comprising a combination of ibuprofen and ketoprofen at a concentration ranging from 0.20% to 4.0% (w/v) of ibuprofen and from 0.0125% to 0.240% (w/v) of ketoprofen, into an isotonic solution. When the composition is administered to an individual's nasal mucosa as a dosage unit, e.g., a 100 μL spray in each nostril for a total of 200 μL, provides an ibuprofen dose ranging from 0.4 mg to 8 mg and a ketoprofen dose ranging from 0.025 to 0.48 mg. The composition is specifically tailored for the intranasal treatment of migraines and other types of headaches.

After the intranasal administration, as described above, the composition allows to reach a plasma Cₘₐₓ of ibuprofen and ketoprofen ranging from 41.4 ng/ml to 827.5 ng/ml and from 1.5 ng/ml to 29.7 ng/ml respectively, in about 15 minutes.

In the same way, the intranasal administration of the composition, results in an AUC_{0-∞} for ibuprofen and ketoprofen ranging from 133.1 to 1921.1 hr*ng/ml and from 0.3 to 45.0 hr*ng/mL.

In a further embodiment, the composition contains at least one of a buffer system, one or more alkalis, one or more isotonizing agents, one or more preservatives, one or more mucoadhesive polymers and one or more cosolvents.

A method of manufacturing the above pharmaceutical composition is also disclosed. The method comprises the steps of dissolving methyl paraben and propylparaben in propylene glycol to produce the paraben concentrated solution, and further diluting the paraben concentrated solution in water resulting in a CFB01 solution; dissolving sodium chloride, potassium chloride, dibasic sodium phosphate and dibasic potassium phosphate in the CBF01 solution, resulting in a Buffer solution; dissolving sodium chloride and sodium bicarbonate, in a mixture of CBF01 solution and water; heating of the solution of sodium chloride, sodium bicarbonate, CBF01 solution, and water to 50° C.±5° C. and dissolving the active ingredients, ibuprofen and ketoprofen; cooling the solution to room temperature and the further dissolving sodium hyaluronate; verifying pH; making up to volume with water; and verifying the osmolarity of the composition.

### BRIEF DESCRIPTION OF THE DRAWINGS

The above and other features, aspects and advantages of the present invention are considered in more detail in relation to the following descriptions of embodiments thereof shown in the accompanying drawings.

FIG. 1. Flow diagram for preparing a sample of a pharmaceutical composition according to one embodiment of the present invention.

FIG. 2. Pharmacokinetic profile (Plasma concentration vs time) of ibuprofen after the administration of 1, 2, 4 and 8 sprays (100 μL) of a pharmaceutical composition containing Ibuprofen 0.2% (w/v), prepared according to one embodiment of the present invention.

FIG. 3. Pharmacokinetic profile (Plasma concentration vs time) of ketoprofen after the administration of 1, 2, 4 and 8 sprays (100 μL) of a pharmaceutical composition containing ketoprofen 0.05% (w/v), prepared according to one embodiment of the present invention.

FIG. 4. Mean pharmacokinetic parameters of ibuprofen and ketoprofen at the four different tested doses.

FIG. 5. Overall results of the exploratory clinical trial in which the preliminary safety and efficacy of a pharmaceutical composition prepared according to one embodiment of the present invention is evaluated.

FIG. 6. Results of the exploratory clinical trial disclosed in FIG. 5, segmented by age.

FIG. 7. Results of the exploratory clinical trial disclosed in FIG. 5, segmented by gender (only women).

FIG. 8. Results of the exploratory clinical trial disclosed in FIG. 5, segmented by gender (only men).

### DETAILED DESCRIPTION OF THE INVENTION

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another. Moreover, it will be appreciated that such a development effort might be complex and time-consuming but would nevertheless be a routine undertaking of engineering for those of ordinary skill in the art having the benefit of this disclosure.

Those of ordinary skill in the art will realize that the following description of the present invention is illustrative only and not in any way limiting. Other embodiments of the invention will readily suggest themselves to such skilled persons having the benefit of this disclosure. Before describing the characteristics of the present invention, it should be understood that the descriptions of specific applications are provided only as representative examples. The present invention is not intended to be limited to the shown embodiments, but it should be given the broadest extent possible, consistent with the principles and characteristics described herein.

### DEFINITIONS AND ABBREVIATIONS

Both in the description and claims of the present invention, the following terminology is used according to the definitions listed below.

“Active ingredient” any component that provides pharmacological activity or other direct effect in the diagnosis, cure, mitigation, treatment, or prevention of disease, or to affect the structure or any function of the body of man or animals. In the context of the present invention, “active ingredient” refers to ibuprofen or ketoprofen separately while the “active ingredients” refers to ibuprofen and ketoprofen together.

“Alkali” any base substance that dissolves in water to produce OH (hydroxide) ions and neutralize acids.

“AUC_{0-∞}”: Area under the curve or AUC is a pharmacokinetic statistic used to describe the total exposure to a drug. More specifically, it is the time-averaged concentration of drug circulating in the body fluid analyzed (normally plasma, blood, or serum). The total AUC or AUC_{0-∞} is the area under the curve from time 0 extrapolated to infinite time.

“About”: when referring to percentages “about” means+/−5%; when referring to quantity, “about” means+/−10 mg or 10 ng; when referring to time “about” means+/−5 minutes.

“Buffer system” is a solution containing a mixture of an acid and its conjugate base, or of a base and its conjugate acid, which can resist changes in its pH when small amounts of an acid or base are added.

“Cₘₐₓ”: A pharmacokinetic measure used to determine drug dosing. Cₘₐₓ, also known as Peak Concentration, is the highest concentration of a drug in the blood, cerebrospinal fluid, or target organ after a dose is given.

“Dosage unit” is the amount of the pharmaceutical composition that would be administered to or taken by one patient at a time.

“Inactive ingredient” any component of a pharmaceutical product other than the active ingredient.

“Intranasal administration” is a route of administration in which drugs are insufflated through the nose. It can be a form of either topical administration or systemic administration, as the drugs thus locally delivered can go on to have either purely local or systemic effects.

“Isotonic” denoting or relating to a solution having the same osmotic pressure as some other solution, especially one in a cell or a body fluid. In the context of the present invention, a solution is considered isotonic when its osmolarity is between 300 mOsm/L and 312 mOsm/L.

“NSAID” nonsteroidal anti-inflammatory drug. Medicines that are widely used to relieve pain, reduce inflammation, and bring down a high temperature, which act by blocking the production of certain body chemicals that cause inflammation.

“Osmolarity” refers to the number of solute particles per 1 L of solvent.

“Pharmacological effect” physiological and/or biochemical changes in the body produced by a drug in therapeutic concentration, as a result of the interaction with a particular receptor.

“Tₘₐₓ”: The time it takes for a drug to reach the maximum concentration (Cₘₐₓ) after administration through a route of administration requiring absorption.

Surprisingly, combining ibuprofen and ketoprofen, two nonselective NSAIDs belonging to the propionic acid derivatives classification, into an isotonic solution for intranasal administration, has shown a significant reduction of the required dose of these active ingredients when compared to the required dose to obtain the desired pharmacological effect when the same ingredients are administered through the oral route. This reduction allows an enhancement in the safety profile of the active ingredients, related to their dose-dependent adverse effects.

A liquid pharmaceutical composition containing a combination of two NSAIDs for intranasal administration, useful in the acute treatment of migraine and other forms of headache is disclosed. In one preferred embodiment, the combination of NSAIDs comprises a mixture ibuprofen and ketoprofen dissolved in an isotonic solution.

More particularly, a solution for intranasal administration in which the amount of each NSAID is significantly lower than the amount required to obtain the desired pharmacological effect when the same active ingredient is administered through the oral route.

In the case of ibuprofen, the minimum approved oral dosage for the treatment of pain in adults is 200 mg. The present invention relates to a solution which, when administered intranasally, provides between 0.4 mg to 8 mg of ibuprofen per dosage unit. This range of dose has not been yet disclosed as an effective dose of ibuprofen in the state of the art.

In the case of ketoprofen, the minimum approved oral dosage for the treatment of pain is 12.5 mg. The present invention relates to a solution which, when administered intranasally, provides between 0.025 mg to 0.48 mg of ketoprofen per dosage unit. This range of dose has not been yet disclosed as an effective dose of ketoprofen in the state of the art.

The combination of these two NSAIDs, administered through this alternative route, has shown a surprising synergic effect which allows a significant reduction of the required dose compared to the required dose to obtain de desired pharmacological effect when the same ingredients are administered through the oral route. The ranges of dose described above, between 0.4 mg to 8 mg of ibuprofen and between 0.025 mg to 0.48 mg of ketoprofen per dosage unit, are equivalent to between 0.2% to 4.0% of the minimum currently approved oral dosage of each of these two NSAIDs, and had not been previously reported as effective in the state of the art.

Owing to the significantly reduced dosages relative to the currently approved doses of both NSAIDs, the intranasal administration of the pharmaceutical composition described herein results in markedly diminished systemic exposure to these active pharmaceutical ingredients. Specifically, the absorption of ibuprofen and ketoprofen through the nasal mucosa, followed by their entry into systemic circulation, achieves levels of systemic exposure ranging from 0.2% to 4.0% of the exposure observed with the minimum approved oral dose of ibuprofen, and from 0.4% to 8.0% of the exposure observed with the minimum approved oral dose of ketoprofen.

The low systemic exposure described above, as shown in the Example 3, as a measure of pharmacokinetic parameters after the intranasal administration of the preparation described herein. There, it can be observed that, for example, the administration of 8 sprays of pharmaceutical composition prepared according to one embodiment of the present, and equivalent to 1.816 mg of ibuprofen and 0.24 mg of ketoprofen, leads to a plasma Cₘₐₓ of 164.7 ng/ml and 24.8 ng/ml for ibuprofen and ketoprofen respectively.

For ibuprofen, a Cₘₐₓ value of 164.7 ng/ml is surprisingly approximately 100 times lower than the expected Cₘₐₓ value for the minimum approved oral dose of 200 mg, which is approximately 17.3 μg/mL, as reported in the literature. In the case of ketoprofen, a Cₘₐₓ value of 24.8 ng/ml is surprisingly approximately 25 times lower than the expected Cₘₐₓ value for the minimum approved oral dose of 12.5 mg, which is approximately 650 ng/ml, according to the published literature.

In general, After the intranasal administration, the composition allows to reach a plasma Cₘₐₓ of ibuprofen and ketoprofen ranging from 36.5 ng/ml to 729 ng/ml and from 2.5 ng/ml to 49.6 ng/ml respectively, in about 15 minutes.

In the same way, the intranasal administration of the composition, results in an AUC_{0-∞} for ibuprofen and ketoprofen ranging from 121.9 to 1697.2 hr*ng/mL and from 3.0 to 74.5 hr*ng/ml.

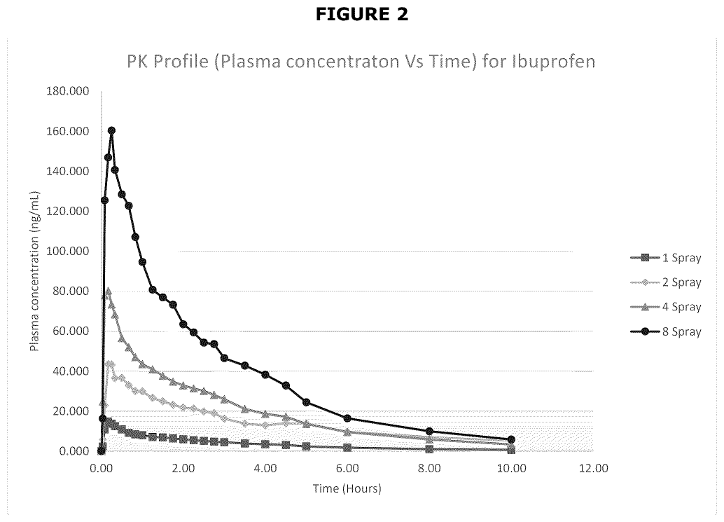

Despite of the low dose, as well as the low systemic exposure to these two NSAIDs, the pharmaceutical composition disclosed herein has proven to be effective in the treatment of migraine and other forms of headache, as shown in the Example 4, and FIGS. 2 to 4 below.

This significant reduction in the dose required to obtain the desired pharmacological effect, accompanied by a corresponding decrease in systemic exposure, allows an enhancement in the safety profile of these active ingredients, since it minimizes their dose-dependent adverse effects and enables their use in a group of patients who do not tolerate NSAIDs at the currently approved therapeutic dosages.

The utilization of the intranasal route of administration also facilitates a reduction in the time required to achieve the maximum plasma concentration (Tₘₐₓ) from approximately two hours, as documented in the literature, to approximately 15 minutes, as demonstrated in Example 3 and illustrated in FIGS. 2 to 4. This decrease in Tₘₐₓ signifies a surprisingly substantial reduction in the onset of action when compared to the administration of the same active ingredients via the oral route.

In addition to ibuprofen and ketoprofen, the pharmaceutical composition includes other inactive ingredients which allows the solubilization of the active ingredients and enhance the efficacy and tolerability of the preparation when administered through the intranasal route. These ingredients are described below.

A buffer system, comprising, for example, a phosphate buffer consisting of the dibasic salt of phosphate with sodium or potassium, the monobasic salt of phosphate with sodium or potassium, or mixtures thereof. The function of this buffer is to adjust the pH of the composition to a physiological pH between 5.5 to 7.5, providing good tolerability of the preparation when it's administered on the nasal mucosa, and reducing the potential for irritation or damage. The phosphate buffer is included at a concentration where the total of the phosphate species adds up to between 0.2 mMol/L to 20.0 mMol/L. It is understood that other buffers can be used in the buffer system as understood by a person of ordinary skill in the art. In some embodiments, the buffer system comprises at least one of dibasic sodium phosphate, dibasic potassium phosphate, monobasic sodium phosphate, monobasic potassium phosphate, or mixtures thereof

Sodium bicarbonate functions as an alkali, reacting with the acid form of the active ingredients to transform them into their salt forms, thus favoring their solubility. It is included at a molar ratio ranging from about 0.9 to about 1.1 in relation to the total content of the active ingredients. Equivalent components include other alkalis such as potassium bicarbonate, sodium or potassium hydroxide, triethanolamine, or mixtures thereof.

Sodium or potassium chloride, or mixtures thereof, function as isotonizing agents. An isotonic solution matches the osmotic pressure of the body's fluids, making it well-tolerated and comfortable for intranasal use. In addition to that, an isotonic solution helps to maintain the integrity of the mucosal membranes, thus preventing the damage to the delicate nasal tissues and supporting optimal drug absorption. It ensures a predictable and consistent pharmacokinetics and enhance the efficacy and safety of the active ingredients. It is included at the concentration required to obtain the isotonicity of the pharmaceutical composition, which is equivalent to an osmolarity ranging from about 300 mOsm/L to about 312 mOsm/L. Equivalent components include dextrose, mannitol, sorbitol, or mixtures thereof.

One or more alkyl parahydroxybenzoates (parabens) function as preservatives to avoid microbial contamination of the pharmaceutical composition. Methyl parahydroxybenzoate is included at a concentration ranging from about 0.05% to about 0.5% and propyl parahydroxybenzoate is included a concentration ranging from about 0.005% to about 0.1%. Equivalent preservatives include benzalkonium chloride at a concentration ranging from about 0.002% to about 0.02%, phenylmercuric nitrate at a concentration ranging from about 0.002% to about 0.02%, thimerosal at a concentration ranging from about 0.002% to about 0.01%, chlorhexidine gluconate at a concentration ranging from about 0.01% to about 0.1%, sorbic acid at a concentration ranging from about 0.05% to about 0.2%, or mixtures thereof.

Sodium hyaluronate functions as a mucoadhesive polymer since it's a naturally occurring polysaccharide with excellent mucoadhesive properties. When applied intranasally, it adheres to the nasal mucosa forming a protective and lubricating film. This mucoadhesive property helps prolong the residence time of the formulation on the nasal mucosa, enhancing the drug's contact and absorption. On the other hand, sodium hyaluronate has exceptional water-retention capabilities. By incorporating it into the pharmaceutical preparation, it helps to moisturize and hydrate the nasal mucosa. Maintaining adequate moisture levels in the nasal cavity enhances patient comfort and promote better drug absorption. Sodium hyaluronate is included at a concentration ranging from about 0.02% to about 2%. Equivalent mucoadhesive include chitosan, carbomers, poloxamers, hydroxypropyl methylcellulose (HPMC), poly (acrylic acid) derivatives, pectin, or mixtures thereof.

Propylene glycol is included as a cosolvent to aid in the solubilization of the active ingredients or any other component of the pharmaceutical preparation. It is included at a concentration ranging from about 0.2% to about 20%. Equivalent cosolvents include polyethylene glycol (PEG), glycerin, ethanol, polyoxyethylene castor oil (Cremophor®), diethylene glycol monoethyl ether (Transcutol®).

Water is the major component of the pharmaceutical preparation and it's especially important for the performance of the formula because it provides the vehicle that sustains all other ingredients. It is included at a concentration ranging from about 80% to 99%.

The preferred pharmaceutical preparation is a clear, colorless and low viscosity solution without the presence of undissolved particles.

The solution and composition described herein can be used for the treatment of migraines and other forms of headaches. In one example of the use of the composition or solution, the composition is administered to an individual prior to the onset of headaches or migraine symptoms. Alternatively, the composition and/or solution can be administered upon the onset of symptoms. Lastly, the composition or solution can be administered after the individual has developed a full headache, migraine, or similar symptoms.

A method of manufacturing the pharmaceutical composition that comprises the dissolution of individual ingredients in their respective solvents, followed by sequential mixing of the resultant solutions in a specified order to prevent precipitation or recrystallization, as detailed in the following steps.

A first step of dissolving methyl parahydroxybenzoate and propyl parahydroxybenzoate in propylene glycol under constant stirring in a vessel, until a clear solution is obtained which is then diluted at a ratio of 1 part in 99 parts of water to obtain a CFB01 solution.

A second step of dissolving a fraction of sodium chloride, potassium chloride, dibasic sodium phosphate, dibasic potassium phosphate in a fraction of CFB01 solution under constant stirring in a second vessel, until a clear solution is obtained.

A third step of dissolving the rest of sodium chloride, the rest of CFB01 solution and sodium bicarbonate in the solution obtained in the second step, under constant stirring until a clear solution is obtained.

A fourth step of dissolving ibuprofen and ketoprofen in the solution obtained in the third step, under constant stirring and heating to 50° C.±5° C. until a clear solution is obtained.

A fifth step consisting of cooling the solution obtained in the fourth step and allowing it to reach room temperature to subsequently dissolve the sodium hyaluronate in it, under constant stirring until a clear solution is obtained.

A final step consisting of adjusting pH to 7±0.5 and make up the volume with water to obtain the final pharmaceutical composition which is then bottled in a proper device for intranasal administration, which delivers, for example, 100 μL per spray, equivalent to 0.227 mg of ibuprofen and 0.03 mg of ketoprofen.

A device containing a solution of ibuprofen at a concentration ranging from 0.20% to 4.0% (w/v) which, when applied to an individual's nasal mucosa, provides an ibuprofen dose ranging from 0.4 mg to 8 mg per dosage unit; and ketoprofen at a concentration ranging from 0.0125% to 0.240% (w/v) which when applied to the nasal mucosa, provides a ketoprofen dose ranging from 0.025 mg to 0.48 mg per dosage unit. The device delivers 100 μL per spray, equivalent to 0.227 mg of ibuprofen and 0.03 mg of ketoprofen.

### Examples

Example 1. Liquid, isotonic pharmaceutical composition containing ibuprofen and ketoprofen for intranasal administration.

An example of the pharmaceutical preparation is shown in Table 1.

| MATERIAL | % | FUNCTION |
|---|---|---|
| Ibuprofen | 0.227 | Active ingredient |
| Ketoprofen | 0.030 | Active ingredient |
| Sodium hyaluronate | 0.050 | Mucoadhesive polymer |
| Sodium chloride | 0.581 | Isotonizing agent |
| Sodium bicarbonate | 0.100 | Alkali |
| Potassium chloride | 0.002 | Isotonizing agent |
| Dibasic sodium phosphate | 0.012 | Buffer agent |
| Dibasic potassium phosphate | 0.002 | Buffer agent |
| Methylparaben | 0.099 | Preservative |
| Propylparaben | 0.011 | Preservative |
| Propylene glycol | 0.439 | Cosolvent |
| Water | C.S.P. 100 mL | Vehicle |

The pH of the solution described above is about 7.4 and the osmolarity is about 303 mOsm/L.

The preparation described above contains 2.27 mg/ml of ibuprofen and 0.3 mg/ml of ketoprofen. It's bottled in a proper device for intranasal administration which delivers 100 μL per spray, equivalent to 0.227 mg of ibuprofen and 0.03 mg of ketoprofen.

The administration is performed by applying 1 spray in each nostril, so the total administered dose is equivalent to 0.454 mg of ibuprofen and 0.06 mg of ketoprofen.

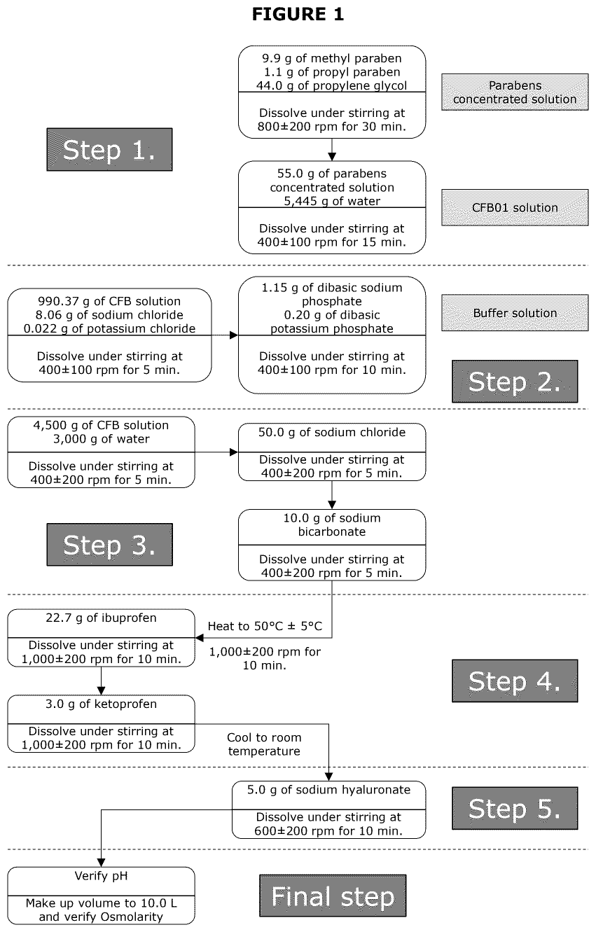

Example 2. Preparation of 10 L of the pharmaceutical composition disclosed in the example 1. A schematic representation of the process is shown in the FIG. 1.

First step: In a 200 ml cleaned vessel, 9.9 g of methyl parahydroxybenzoate and 1.1 g of propyl parahydroxybenzoate were dissolved in 44.0 g of propylene glycol under stirring (stirrer rpm 800±200) for 30 minutes, until a clear solution was obtained. This solution was labeled as “Parabens concentrated solution.”

In a 7 L cleaned vessel, 5,445 g of water were added. The water was kept under stirring (stirrer rpm 400±100) and then paraben concentrated solution was slowly added, stirring for 15 minutes until a clear solution was obtained. This solution was labeled as “CFB01 solution.”

Second step: In a 2 L clean vessel, 990.37 g of CFB01 solution were added, The CBF01 solution was kept under stirring (stirrer rpm 400±100) and then 8.06 g of sodium chloride and 0.22 g of potassium chloride were slowly added, stirring for 5 minutes until a clear solution was obtained. Then 1.15 g of dibasic sodium phosphate and 0.20 g of dibasic potassium phosphate were slowly added to the above solution, stirring for 10 additional minutes until a clear solution was obtained. This solution was labeled as “Buffer solution.”

Third step: In a 15 L preparation tank 4,500 g of CFB01 solution were diluted in 3,000 g of water under stirring (stirrer rpm 400±200) for 5 minutes until a clear solution was obtained. Then 50 g of sodium chloride were slowly added to the above solution, stirring for 5 additional minutes until a clear solution was obtained. Then 10 g of sodium bicarbonate were slowly added to the above solution, stirring for 5 additional minutes until a clear solution was obtained.

Fourth step: The solution obtained in the third step was heated at 50° C.±5° C. under stirring (stirrer rpm 1,000±200) for 10 minutes. Then 22.7 g of ibuprofen were slowly added to the above solution while the temperature was maintained at 50° C.±5° C. and stirring for 10 additional minutes until a clear solution was obtained. Then 3.0 g of ketoprofen were slowly added to the above solution while the temperature was maintained at 50° C.±5° C. and stirring for 10 additional minutes until a clear solution was obtained.

Fifth step: The solution obtained in the fourth step was allowed to reach room temperature and then 5.0 g of sodium hyaluronate were added were slowly added to the solution under stirring (stirrer rpm 600±200) for 10 minutes until a clear solution was obtained.

Final step: The pH was verified to be in the range of 7.0 to 7.5 and the volume was made up to 10 L with water. Then, the osmolarity was verified to be in the range of 300 to 312 mOsm/L and the product bottled in 10 mL devices for intranasal administration.

Example 3. Pharmacokinetic study to evaluate the bioavailability of the pharmaceutical composition prepared according to the present disclosure.

An open-label, randomized, single-dose, bioavailability study of ibuprofen 0.2% (w/v)+ and ketoprofen 0.05% (w/v) nasal spray (for four different doses) in healthy adult human subjects under fasting conditions.

32 healthy adult human subjects were enrolled in the study with 4 dosing groups (8 subjects per group), with the age limit of 18-45 years (both inclusive) with a Body Mass Index (BMI) ranging between 18.50 Kg/m² and 30.00 Kg/m² (both inclusive).

Dose 1 (A): One spray [0.1 mL per spray]−One spray of ibuprofen 0.2% (w/v)+ketoprofen 0.05% (w/v) nasal spray was administered in either right or left side of the nostril. [Total Dose: 0.2 mg of ibuprofen+0.05 mg of ketoprofen]

Dose 2 (B): Two sprays [0.1 mL per spray]−One spray of ibuprofen 0.2% (w/v)+ketoprofen 0.05% (w/v) nasal spray was administered in each nostril. [Total Dose: 0.4 mg of ibuprofen+0.1 mg of ketoprofen]

Dose 3 (C): Four sprays [0.1 mL per spray]−Two sprays of ibuprofen 0.2% (w/v)+ketoprofen 0.05% (w/v) nasal spray were administered in each nostril. [Total Dose: 0.8 mg of ibuprofen+0.2 mg of ketoprofen]

Dose 4 (D): Eight sprays [0.1 ml per spray]−Four sprays of ibuprofen 0.2% (w/v)+ketoprofen 0.05% (w/v) nasal spray were administered in each nostril. [Total Dose: 1.6 mg of ibuprofen+0.4 mg of ketoprofen]

In total, 25 venous blood samples (6 mL each) were collected from each subject at pre-dose (00.00 hour) within 1.00 hour prior to dosing and at 0.033, 0.083, 0.167, 0.25, 0.33, 0.50, 0.67, 0.83, 1.00, 1.25, 1.50, 1.75, 2.00, 2.25, 2.50, 2.75, 3.00, 3.50, 4.00, 4.50, 5.00, 6.00, 8.00 and 10.00 hours post-dose within the window period of +2 minutes.

Blood samples were centrifuged at about 4000 rpm for 10 minutes at 4±2° C. to separate the plasma. The resulting plasma was equally transferred into 2. These aliquots were stored frozen, upright in a freezer within 90 minutes of blood collection time of each sample at a temperature of −70° C. #15° C. until they were analyzed.

The concentration of ibuprofen and ketoprofen in plasma samples were analyzed using LC-MS/MS method. The Lower Limit of Quantification (LLOQ) for ibuprofen was 1.0 ng/ml and for ketoprofen was 0.1 ng/ml. Sample values below the LLOQ were reported as Below Quantification Limit (BQL). Pharmacokinetic parameters were calculated using Phoenix® WinNonlin® software (version: 8.3). The following parameters were calculated:

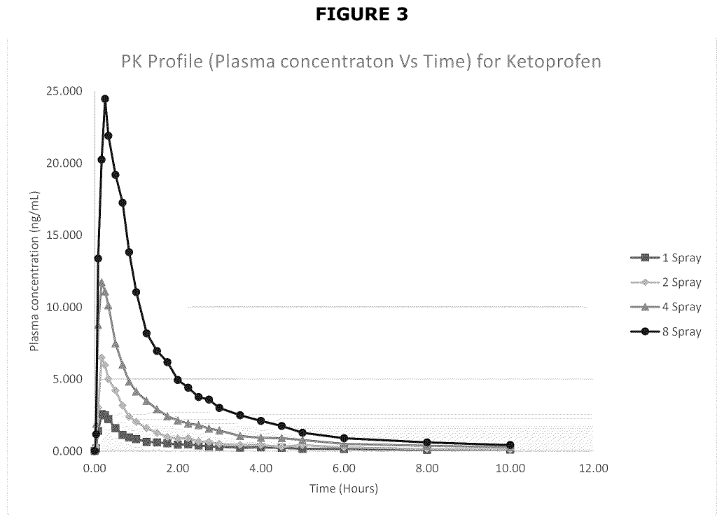

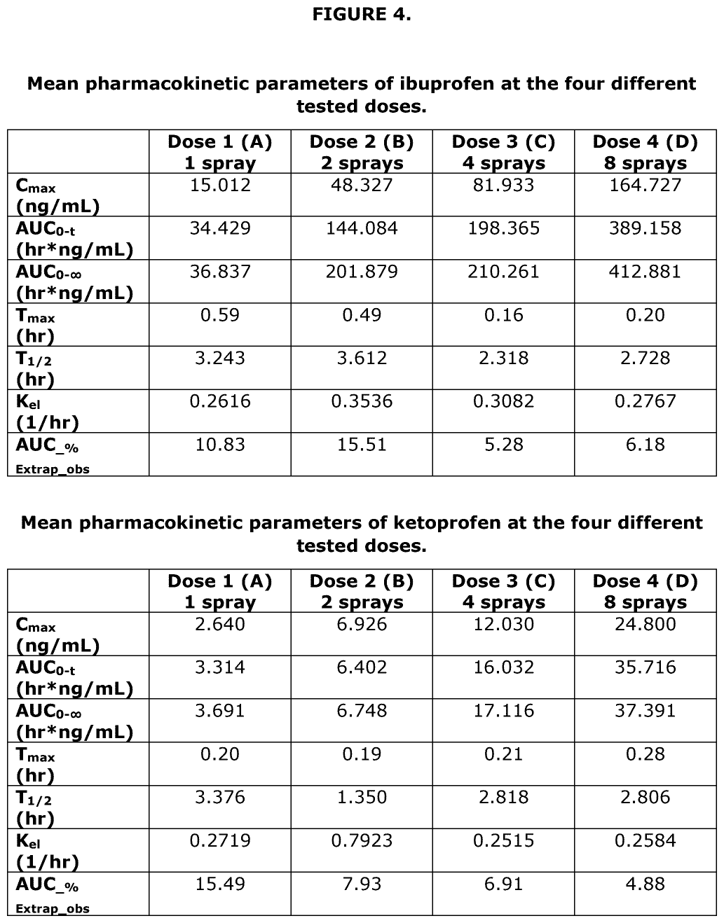

Primary pharmacokinetic parameters: Cₘₐₓ, AUC₀₋ₜ and AUC_{0-∞} Secondary pharmacokinetic parameters: tₘₐₓ, t_{1/2}, Kₑₗ and AUC__{% Extrap_obs}. Obtained pharmacokinetic profiles (Plasma concentration Vs Time) for ibuprofen and ketoprofen are shown in FIG. 2 and FIG. 3, respectively. The mean pharmacokinetic parameters of ibuprofen and ketoprofen at the four different tested doses are shown in FIG. 4.

Example 4. Exploratory clinical trial to evaluate the preliminary safety and efficacy of the pharmaceutical composition prepared according to the present disclosure in the acute treatment of migraine.

An open-label, multicenter exploratory trial to evaluate the clinical efficacy and safety of ibuprofen 0.227%+ketoprofen 0.03% solution for intranasal administration in the acute treatment of migraine headaches in adult subjects was conducted.

Potential subjects were identified by the investigators during their routine practice, and these identified subjects were informed about the study to see if they had an interest to participate in it. Interested subjects were further evaluated for eligibility. Eligible subjects with established diagnosis of migraine headache were enrolled in the study.

Inclusion criteria included: 1) Male or non-pregnant, non-lactating female, with the age limit of ≥18 years to ≤65 years; 2) subject able to communicate effectively and provide written informed consent; 3) female subject of childbearing potential NOT being pregnant or breastfeeding; 4) previous clinical diagnosis of migraine headaches by a qualified physician; 5) subject exhibiting signs of an acute migraine attack on presentation to investigating physician; 6) subjects willing to abide by the study requirements and give subjective feedback after test product is administered.

Exclusion criteria included: 1) Pregnancy or breastfeeding or planning to become pregnant during the study period; 2) use of medication to treat migraine within 12 hours prior to seeing investigator; 3) history of alcohol or other substance abuse within the last one year; 4) known hypersensitivity to ibuprofen, ketoprofen or any other NSAIDs or components of the formulation; 5) inability to understand the requirements of the study and the relative information or to comply with the study protocol; 6) any other condition of the subject, which, in the opinion of the investigator, which is likely to interfere with the study treatment and assessments; 7) other severe acute or chronic medical or chronic medical or psychiatric conditions or laboratory abnormalities that may increase the risk associated with study participation or study drug administration or may interfere with the interpretation of study results and, in the judgement of the investigator, would make the subject inappropriate for entry into the trial.

A total of 20 subjects (6 males and 14 females) were enrolled in the study. The age of the enrolled subjects ranged from 17 to 61 (median 35.5) years. A single application (100 μL) of the test product in each nostril were performed in the doctor's office. If the patient had not experienced relief in 10 minutes another application was performed to the subject.

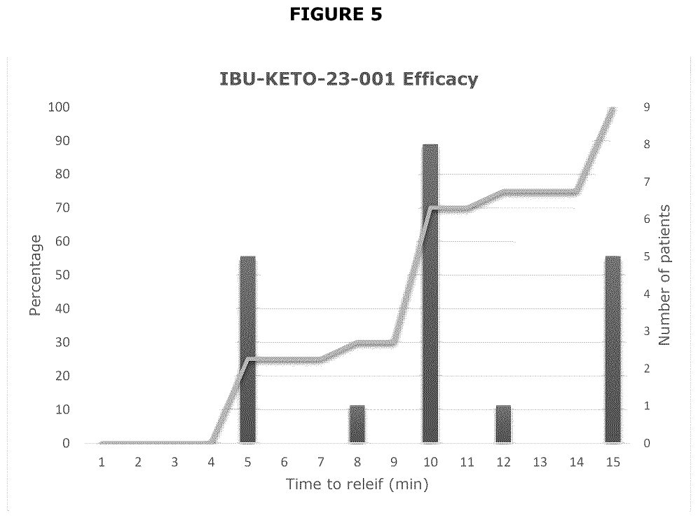

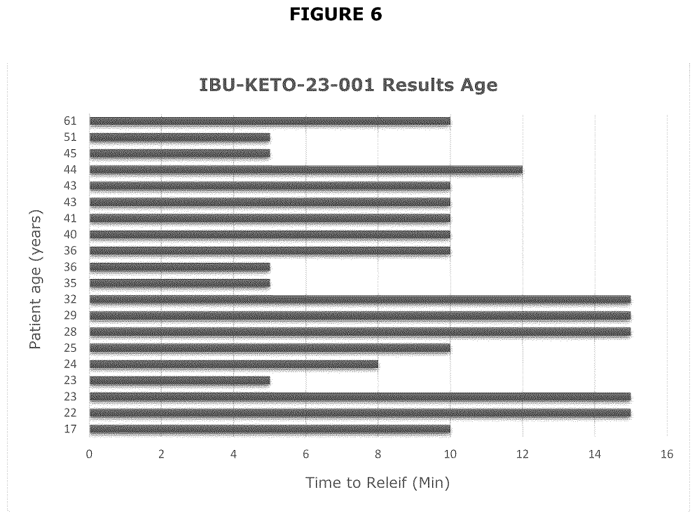

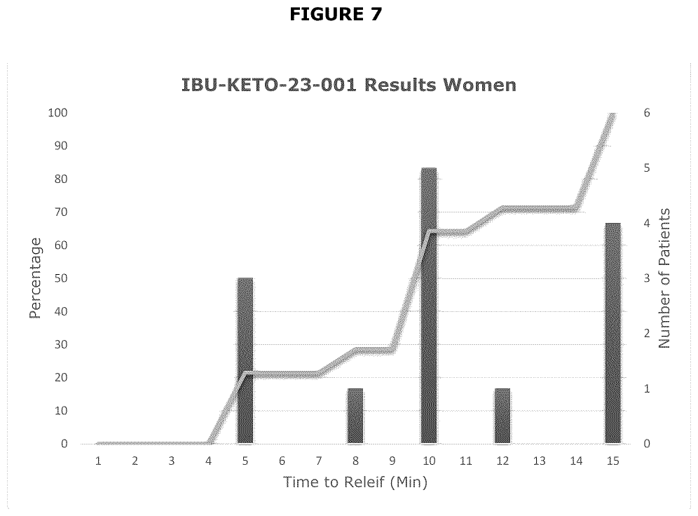

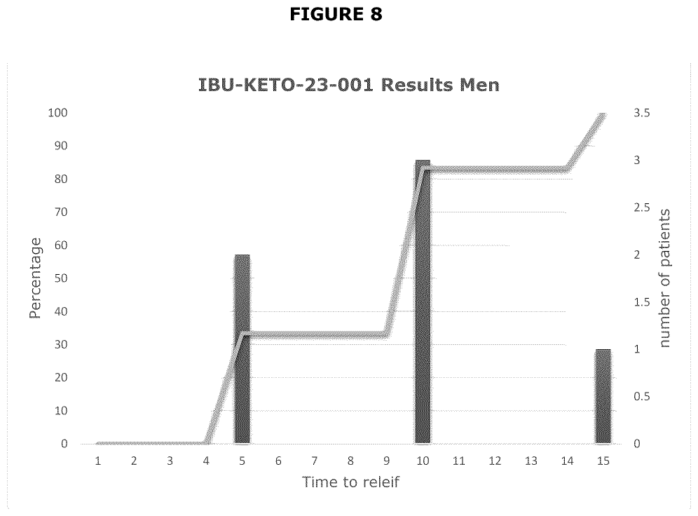

The primary efficacy endpoint of the study is the reduction symptoms of migraine attack in study subjects. Reduction in patient symptoms is defined as the subjective reduction of symptoms from the patient's perspective. From the efficacy point of view, all patients reported a reduction or elimination of the migraine symptoms within 15 minutes of treatment administration. The results are shown in detail in the FIGS. 5 to 8. From the safety point of view, none of the patients reported side effects.

What is claimed is:

1. A pharmaceutical composition for intranasal administration and treatment of migraines and other forms of headaches, comprising:
ibuprofen at a concentration ranging from 0.20% to 4.0% (w/v) which, when applied to an individual's nasal mucosa, provides an ibuprofen dose ranging from 0.4 mg to 8 mg per dosage unit; and
ketoprofen at a concentration ranging from 0.0125% to 0.240% (w/v) which when applied to the nasal mucosa, provides a ketoprofen dose ranging from 0.025 mg to 0.48 mg per dosage unit.

2. The pharmaceutical composition of claim 1, which when applied to the nasal mucosa, produces plasma Cₘₐₓ of ibuprofen and ketoprofen ranging from 36.5 ng/ml to 729 ng/ml and from 2.5 ng/ml to 49.6 ng/ml respectively, in about 15 minutes.

3. The pharmaceutical composition of claim 1, which when applied to the nasal mucosa, produces an AUC_{0-∞} for ibuprofen and ketoprofen ranging from 121.9 to 1697.2 hr*ng/ml and from 3.0 to 74.5 hr*ng/ml.

4. The pharmaceutical composition of claim 1, further comprising at least one of a buffer system, one or more alkalis, one or more isotonizing agents, one or more preservatives, one or more mucoadhesive polymers and one or more cosolvents.

5. The pharmaceutical composition of claim 1, where the pharmaceutical composition is an isotonic solution in water, with a pH ranging between 5.5 to 7.5.

6. The pharmaceutical composition of claim 4, wherein the buffer system comprises at least one of dibasic sodium phosphate, dibasic potassium phosphate, monobasic sodium phosphate, monobasic potassium phosphate, or mixtures thereof.

7. The pharmaceutical composition of claim 4, wherein the one or more alkalis is selected from the group consisting of sodium bicarbonate, potassium bicarbonate, sodium hydroxide, potassium hydroxide, triethanolamine, or mixtures thereof.

8. The pharmaceutical composition of claim 4, wherein the one or more isotonizing agents is selected from the group consisting of sodium chloride, potassium chloride, sodium hydroxide, dextrose, mannitol, sorbitol, or mixtures thereof.

9. The pharmaceutical composition of claim 4, wherein the one or more preservatives is selected from the group consisting of methyl parahydroxybenzoate, propyl parahydroxybenzoate, benzalkonium chloride, phenylmercuric nitrate, thimerosal, chlorhexidine gluconate, sorbic acid, or mixtures thereof.

10. The pharmaceutical composition of claim 4, wherein the one or more mucoadhesive polymers is selected from the group consisting of sodium hyaluronate, chitosan, carbomers, poloxamers, hydroxypropyl methylcellulose (HPMC), poly (acrylic acid) derivatives, pectin, or mixtures thereof.

11. The pharmaceutical composition of claim 4, where the one or more cosolvents is selected from the group consisting of propylene glycol, polyethylene glycol (PEG), glycerin, ethanol, polyoxyethylene castor oil, diethylene glycol monoethyl ether, or mixtures thereof.

12. A method of manufacturing a pharmaceutical composition for intranasal administration and treatment of migraines and other forms of headaches, comprising the dissolution of individual ingredients in their respective solvents, followed by sequential mixing of the resultant solutions in a specified order to prevent precipitation or recrystallization, as detailed in the following steps:
dissolving methyl paraben and propylparaben in propylene glycol to produce a paraben concentrated solution, and further diluting the paraben concentrated solution in water resulting in a CFB01 solution;
dissolving the isotonizing agents, as well as the component of the buffer system in solution CBF01, resulting in a Buffer solution;
dissolving sodium chloride and sodium bicarbonate, in a mixture of CBF01 solution and water;
heating of the solution of sodium chloride, sodium bicarbonate, CBF01 solution, and water to 50° C.±5° C. and dissolving one or more active ingredients, ibuprofen and ketoprofen;
cooling the solution to room temperature and the further dissolving sodium hyaluronate;
verifying pH;
making up to volume with water; and
verifying the osmolarity of the composition.

13. The method of claim 12, further comprising the step of packaging the composition in a device suitable for intranasal administration.

14. The method of claim 13, wherein the device delivers 100 μL per spray, equivalent to 0.227 mg of ibuprofen and 0.03 mg of ketoprofen.

15. A device for the delivery of a pharmaceutical composition for intranasal administration and treatment of migraines and other forms of headaches, comprising:
ibuprofen at a concentration ranging from 0.20% to 4.0% (w/v) which, when applied to an individual's nasal mucosa, provides an ibuprofen dose ranging from 0.4 mg to 8 mg per dosage unit; and
ketoprofen at a concentration ranging from 0.0125% to 0.240% (w/v) which when applied to the nasal mucosa, provides a ketoprofen dose ranging from 0.025 mg to 0.48 mg per dosage unit.

16. The device of claim 15, wherein the device delivers a dosage of 100 μL per spray, equivalent to 0.227 mg of ibuprofen and 0.03 mg of ketoprofen.

17. The device of claim 15, wherein the solution further comprises at least one of a buffer system, one or more alkalis, one or more isotonizing agents, one or more preservatives, one or more mucoadhesive polymers and one or more cosolvents.

18. The device of claim 15, where the pharmaceutical composition is an isotonic solution in water, with a pH ranging between 5.5 to 7.5.

19. The device of claim 17, wherein the buffer system comprises at least one of dibasic sodium phosphate, dibasic potassium phosphate, monobasic sodium phosphate, monobasic potassium phosphate, or mixtures thereof.
